(19) United States
(12) Patent Application Publication
(10) Pub. No.: US 2018/0296163 A1
(43) Pub. Date: Oct. 18, 2018

(54) Altering Physiological Signals Based On Patient Movement

(71) Applicant: Google Inc., Mountain View, CA (US)

(72) Inventors: Brian Derek DeBusschere, Los Gatos, CA (US); Jeffrey L. Rogers, San Carlos, CA (US)

(21) Appl. No.: 14/809,901

(22) Filed: Jul. 27, 2015

(51) Int. Cl. A61B 5/00; A61B 5/024; A61B 5/0205; A61B 5/08; A61B 5/026; A61B 5/11; A61B 5/021

(52) U.S. Cl. CPC A61B 5/721; A61B 5/024; A61B 5/02405; A61B 5/02141; A61B 5/08; A61B 5/026; A61B 5/1102; A61B 5/0205

(57) ABSTRACT

This document describes ways in which to alter physiological signals to address corrupt, noisy, or otherwise faulty data. By so doing, accuracy and robustness in sensing and assessing a patient's cardiovascular health can be improved. These improved assessments permit better measures of health, such as relevant hemodynamics understood by heart rates, heart rate variability, cardiac arrhythmias, blood pressures, pulse-wave velocities, arterial stiffness, cardiac valve timing, thoracic fluids, ballistocardiogram force, photo-plethysmograms, blood oxygenation, and pressure-volume loops.

### BACKGROUND

Cardiovascular disease is the leading cause of morbidity and mortality worldwide. At the same time, this chronic disease is largely preventable. Medical science knows how to save most of these lives by removing the major risk factors of smoking, diabetes, and hypertension. In addition, many people are told just what they need to do to reduce these risk factors—stop smoking, reduce sugar intake, eat healthier, reduce alcohol intake, increase cardiovascular exercise, lose weight, and, if needed, take blood-pressure medication. Nevertheless, many people do not follow this good advice. Because of this, millions of people needlessly die from cardiovascular disease.

People do not follow this good medical advice because they think they are different, they do not want to change their behaviors that are causing the disease, or they do not know what to change in their particular case. When a physician tells them that they are at risk from heart disease because they are overweight, for example, many people know that this judgment is not necessarily specific to them—it is based on averages and demographics. So being a particular weight may not negatively affect a particular patient's heart. Further, a lack of feedback that their behavior is harming their heart results in a lack of incentive for them to change their behavior.

This lack of incentive to follow good advice can be addressed by monitoring the state of the patient's cardiovascular system both on occasion or over time to show trends in heart health. Hard, physiological data often motivates patients to modify their behavior, such as data indicating that their heart shows measurable signs of heart disease. Unfortunately, current methods for measuring heart health can be inconvenient, inaccurate, and expensive. Simple home monitor products exist for measuring heart rate and blood pressure, but long-term user compliance is a problem due to inconvenience, and accuracy can easily suffer from patient misuse, signal noise, or signal corruption for all but the simplest monitors. More advanced cardiovascular monitoring, such as heart rate variability, arterial stiffness, cardiac output, and atrial fibrillation, involve expensive and time-consuming trips to a medical facility for a skilled assessment and, even when performed, often suffers from corrupt, noisy, or otherwise faulty physiological signals.

### SUMMARY

This document describes ways in which to alter physiological signals to address corrupt, noisy, or otherwise faulty data. By so doing, accuracy and robustness in sensing and assessing a patient's cardiovascular health can be improved. These improved assessments permit better measures of health, such as relevant hemodynamics understood by heart rates, heart rate variability, cardiac arrhythmias, blood pressures, pulse-wave velocities, arterial stiffness, cardiac valve timing, thoracic fluids, ballistocardiogram force, photo-plethysmograms, blood oxygenation, and pressure-volume loops.

The techniques disclosed in this document use various sensors to sense the effects of cardiovascular hemodynamics. As noted, one challenge associated with using physiological sensors is signal corruption. The techniques determine if physiological signals are corrupt based on patient movement, such as respiration or major body movements. By so doing, these corrupt physiological signals can be altered, removed, or otherwise addressed to improve assessment of a patient's heart health.

Through these techniques, health assessments using physiological signals can be improved, whether at one particular time or over time to provide a health trend. Trends can aid a patient by helping them know if the effort they are expending to improve their heart health is actually making a difference. Further, negative trends or conditions, such as cardiac irregularities or some asymmetries can be found that can spur people to improve their health or to get medical attention. By so doing, these techniques may save many people from dying of heart disease.

This summary is provided to introduce simplified concepts concerning the techniques, which are further described below in the Detailed Description. This summary is not intended to identify essential features of the claimed subject matter, nor is it intended for use in determining the scope of the claimed subject matter.

### BRIEF DESCRIPTION OF THE DRAWINGS

Embodiments of techniques and devices for sensing cardiovascular health and altering physiological signals based on patient movement are described with reference to the following drawings. The same numbers are used throughout the drawings to reference like features and components:

FIG. 1 illustrates an example environment in which the techniques can be implemented.

FIG. 2 illustrates an example computing device of FIG. 1.

FIG. 3 illustrates example physiological/motion sensors of FIG. 1.

FIG. 4 illustrates a method for altering physiological signals based on patient movement, including to assess hemodynamic characteristics for a patient.

FIG. 5 illustrates a sensing milieu in which a female patient is measured by physiological sensors and a motion sensor.

FIG. 6 illustrates various captured physiological signals and a motion data signal for the patient of FIG. 5.

FIG. 7 illustrates a method for altering physiological signals based on patient movement, including by determining that a segment of the physiological signal is corrupt.

FIG. 8 illustrates a time window for the arm movement of FIGS. 5 and 6, along with a corresponding segment of a physiological signal.

FIG. 9 illustrates an example device in which techniques may be implemented that alter physiological signals based on patient movement.

### DETAILED DESCRIPTION

### Overview

This document describes techniques and devices for altering physiological signals based on patient movement. These altered signals permit better measurement of a patient's health.

Consider non-invasive, automated monitors of cardiovascular health. These types of monitors tend to have excellent user compliance when used in the patient's home. These types of monitors, however, tend to have signals with poor signal-to-noise ratios, that are at least partially corrupt, and that include artifacts. The less noticeable or invasive that they are made, the more their signal quality often suffers. As a result, there is decreased diagnostic confidence in the automated measurements from these sensors, reducing their utility and deployment.

By way of example, consider an integrated floormat capable of sensing data to create an electrocardiograph (ECG) and ballistocardiogram (BCG). The floormat offers the advantage of an inconspicuous measurement device that is much less disruptive to the patient than standard ECG electrodes or blood-pressure cuffs. Beyond the standard vitals of heart rate, heart rate variability, and respiration rate, the ECG and BCG can nominally be combined to monitor the timing of important cardiac events, such as contraction and aortic valve opening. They can also be combined with a photo-plethysmogram (PPG) to measure pulse transit time, allowing for an estimation of pulse wave velocity, which correlates with arterial blood pressures. This type of monitor, however, often has low signal quality and susceptibility to artifacts, such as a muscle's EMG corrupting the ECG or physical body motion corrupting BCG.

The techniques, however, can determine a patient's movement during this monitoring, thereby enabling alteration of these physiological signals. A camera may track a patient's arm movement sufficient to calculate a potential muscle EMG corruption, or a shift in the patient's body standing on the floormat sufficient to indicate a possible BCG corruption as the patient's weight shifts from one side to another. The techniques, with this physical movement matched to the physiological signals from the floormat, can improve the quality of the physiological signals and therefore of the health assessment that is based on those physiological signals.

These are but a few examples in which altering physiological signals based on patient movement can be performed, other examples and details are provided below. This document now turns to an example environment, after which example physiological sensors and methods, and an example computing system are described.

### Example Environment

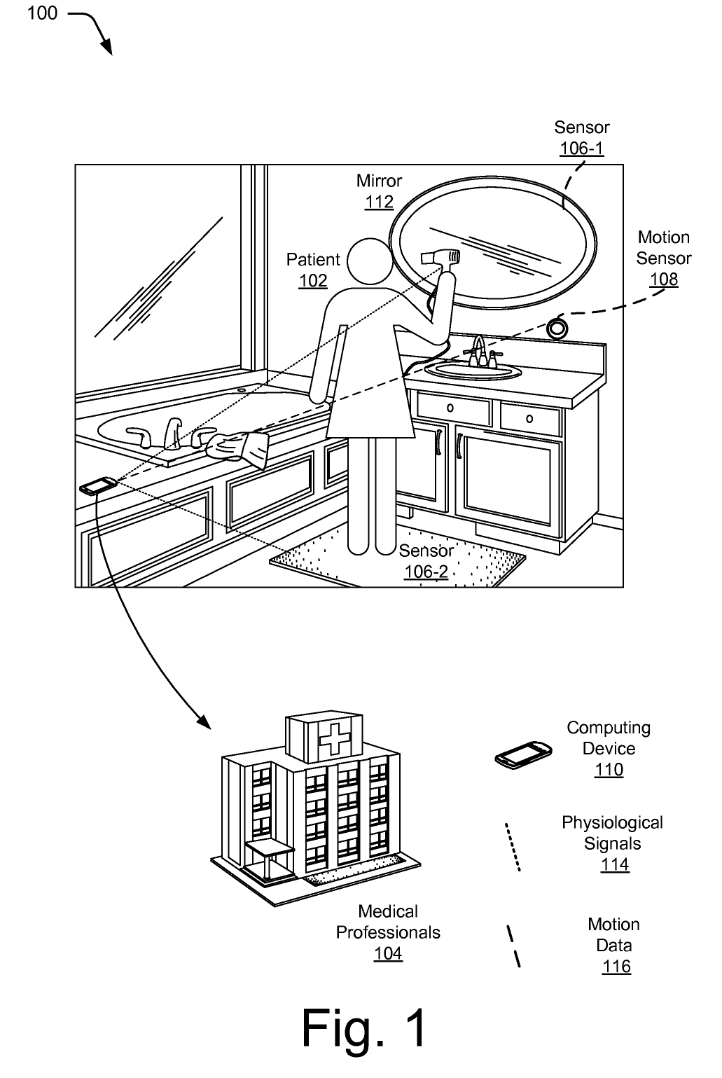

FIG. 1 is an illustration of an example environment 100 in which physiological signals are altered based on patient movement. Environment 100 illustrates a patient 102 and a medical professional 104, family member, or other caretaker that, in some cases, will receive results of the health monitoring. This example employs physiological sensors 106 and a motion sensor 108, optionally in communication with computing device 110, a hyperspectral sensor 106-1, which is located within mirror 112, and a pressure and electrical-sensing mat 106-2.

Physiological signals 114 are provided by each of physiological sensors 106 to some computing device. These signals are effective to measure a hemodynamic characteristic of a patient, as such as a pulse-wave velocity or pressure waves representing blood flow through an artery or vein.

As shown, physiological signals 114 are passed from sensors 106 to computing device 110, though they may instead be integral with a computing device. Computing device 110 then performs some or all of the techniques, or passes those physiological signals to some other computing device, such as a remote server through a communication network (not shown).

As shown with this example environment 100, a sensing milieu (e.g., physiological sensors 106 in patient 102's bathroom) in which a patient lives can be used that are capable of determining a hemodynamic characteristic of a human cardiovascular system. This sensing milieu is capable of non-invasively and remotely determining this hemodynamic characteristic and trends thereof This sensing milieu senses various regions of the patient, which can then be compared, synchronized, aggregated, averaged, and so forth. These hemodynamic characteristics can be represented by cardiovascular asymmetries (e.g., due to a stoke), cardiac irregularities (e.g. atrial fibrillation), blood pressure, pulse-wave velocity, waveforms of circulating blood, photo-plethysmograms (PPG), ballistocardiograms, and pressure-volume loops, to name a few.

Each of these physiological sensors 106 provide physiological signals 114 that include some error, noise, and so forth. Motion sensor 108 also provides motion data 116 to computing device 110. As noted above, the techniques permit alteration of these signals to improve medical assessment of patients based on movement of patient 102.

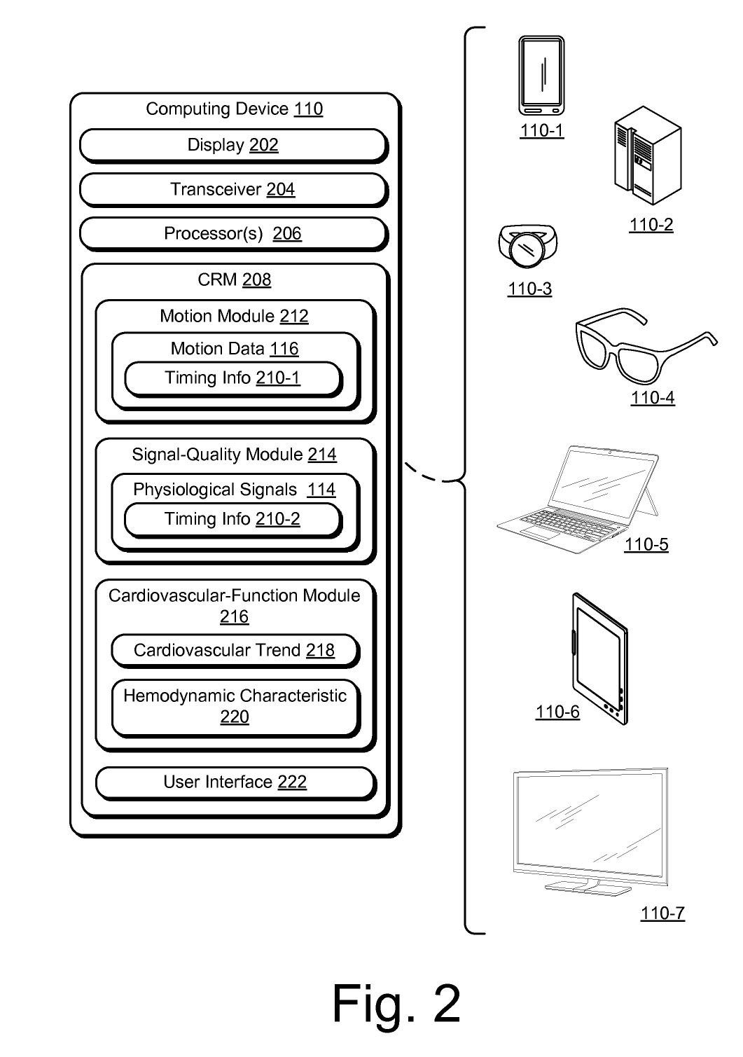

With regard to computing device 110 of FIG. 1, consider a detailed illustration in FIG. 2. Computing device 110 can be one or a combination of various devices, here illustrated with seven examples: a smartphone 110-1, a server 110-2, a computing watch 110-3, computing spectacles 110-4, a laptop 110-5, a tablet computer 110-6, and a desktop 110-7, though other computing devices and systems, such as one of physiological sensors 106 that includes computing capabilities, a netbook, or a set-top box may also be used. As noted above, in some embodiments the techniques operate, in whole or in part, through a remote device such as server 110-2. In such cases, some computing can be forgone locally, e.g., through a communication device having limited computing operations or even directly from physiological sensors 106 and/or motion sensor 108 to server 110-2.

Computing device 110 may include or be able to communicate with a display 202 (six are shown in FIG. 2), though a display is not required. Computing device 110 includes or is able to communicate with a transceiver 204, one or more processors 206, and computer-readable storage media 208 (CRM 208). Transceiver 204 is capable of sending and receiving data directly or through a communication network, such as physiological signals 114 from physiological sensors 106 through a local area, wide area, cellular, or near-field network.

CRM 208 includes motion data 116 and physiological signals 114. Motion data 116 is received from motion sensor 108 and physiological signals 114 from sensors 106, as shown in FIG. 1. Motion data 116 and physiological signals 114 include timing information 210-1 and 210-2, respectively. Timing information 210 indicates a time or mark sufficient to chronologically synchronize physical movements of patient 102 (determined from motion data 116) to physiological signals 114 for patient 102.

CRM 208 also includes a motion module 212 and a signal-quality module 214. Motion module 212 is configured to determine, based on the captured motion data (motion data 116), physical movements of patient 102. This motion data 116 can be captured by various types of motions sensors 108, such as those determining movement through SONAR (SOund Navigation And Ranging), infrared, radar, an optical camera capturing multiple still images over time, and so forth. Further, this motion data 116 can be received with timing information 210-1 or timing can be determined for the data based on when it is received, latency due to capture, processing, and transmission times, and so forth.

Signal-quality module 214 is configured to receive physiological signals from physiological sensors oriented to the patient, such as physiological signals 114 captured for patient 102 of FIG. 1 and chronologically synchronize physical movements for the patient with the physiological signals. Thus, based on physical movements determined by motion module 212, signal-quality module 214 can synchronize those movements with physiological signals 114. Once synchronized, signal-quality module 214 then alters physiological signals 114 based on the physical movements.

Signal-quality module 214 may alter physiological signals in various manners. Signal-quality module 214 may down-weight the physiological signals based on the physical movement determined to be a large physical movement of the patient. These large physical movements often have a negative effect on the quality, or the usefulness, of the signal. Thus, in some cases a person walking, or shifting his weight, or moving his arms up and down causes the signal to be inaccurate, noisy, and so forth. These signals can be down-weighted when such movements are taking place, thereby reducing their value for multiple signals over multiple similar events, like heartbeats. In some other cases, the signal is still of high quality, but the usefulness is reduced (in some it is increased, discussed below). A blood pressure reading via a physiological signal, for example, may be accurate but less useful when the reading is made when a person is in the process of standing up, as often a measurement is desired at a relatively steady or resting state of the patient.

Similarly, signal-quality module 214 may alter physiological signals by removing parts of the physiological signals where the physical movement of the patient is disruptive. Assume, for example, that for a particular cardiovascular event, like a single heartbeat, that the patient makes a jerky or major body movement. This movement may render the physiological signals for that heartbeat not reliable or useful for measuring the patient's heart health.

Signal-quality module 214 may alter physiological signals based on the patient's respiration. Thus, after determining that the physical movement represents the patient breathing, signal-quality module alters the physiological signals based on this known, well-understood movement. Respiration and other common movements have a known effect, either for patients generally, or learned over prior physiological signals and respirations being chronologically synchronized and physiological signal differences based on respiration learned.

In some cases these alterations compensate for an impact of a physical movement. This compensation enable the physiological signals to be correlated with other physiological signals having a same type.

In addition to the above alterations, signal-quality module 214 can alter after first determining a portion of the physiological signal that is likely to include noise or signal error. In such a case, the portion of the physiological signal is then chronologically synchronized to the physical movements for the patient. When those physical movements for the patient are determined likely to cause noise or signal error, the alteration reduces a weight of, corrects, or removes the portion of the physiological signal.

As noted above, some physical movements can be useful in assessing a patient's health. In such a case, signal-quality module 214 annotates the physiological signals based on the physical movement determined to match one of a set of previously determined physical movements useful in assessing cardiovascular health. Assume, for example, that bowel movements are useful in assessing cardiovascular health. A physical movement consistent with this bodily function is determined, and physiological signals matched to the bodily function are annotated. This permits later analysis, or current analysis of the patient's health, oftentimes in a manner that is either more helpful, or simply offering different data, than low-activity states of the patient. Other physical movements can also be annotated, such as a patient moving up her arms, singing, breathing, walking, and so forth.

CRM 208 also includes cardiovascular-function module 216, which is configured to use altered physiological signals 114 to determine a health condition or trend. In the case of trends, cardiovascular-function module 216 uses physiological signals 114 (altered or unaltered) that are associated with particular dates to determine cardiovascular trends 218 in a hemodynamic characteristic 220. CRM 208 also includes or has access to a user interface 222, that, while not required, can be used to present determined trends, health, and medical advice to patient 102.

Generally, cardiovascular-function module 216 is capable of determining, based on altered and unaltered physiological signals 114, a hemodynamic characteristic of a cardiovascular system of a patient, such as patient 102 of FIG. 1. With this hemodynamic characteristic, cardiovascular-function module 216 may alert patient 102 or medical professionals 104 or family members/caretakers of a negative health condition needing immediate care, for example. Medical professional 104, or a specialized machine intelligence, can schedule an in-person appointment or remotely adjust patient care through changes in medication or lifestyle. Cardiovascular-function module 216 is also configured to determine trends based on the current hemodynamic characteristic and prior-determined hemodynamic characteristics.

More specifically, cardiovascular-function module 216 is capable of receiving and using physiological signals 114, which indicates a patient's skin color, displacement, heart rate, blood pressure, and various other factors. This data may come from single or multiple physiological sensors 106 measuring the same or different locations on the patient's body. With this data, cardiovascular-function module 216 can determine pulse pressure waveforms and asymmetries in a patient's cardiovascular system. With this data and a circulatory distance between data from different regions of the patient, cardiovascular-function module 216 can determine a pulse-wave velocity and various simple or highly sophisticated measures of cardiovascular health, including charts of blood pressure, a ballistocardiogram, a photo-plethysmogram (PPG), and pressure-volume loops.

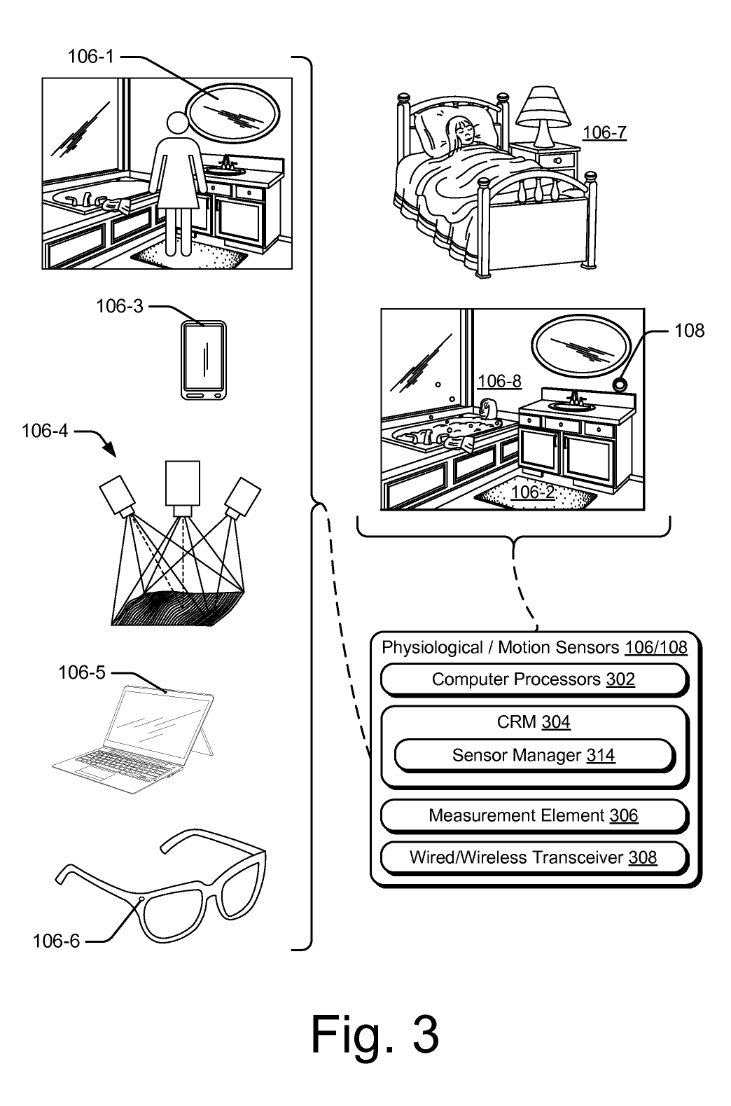

With regard to physiological sensors 106, two examples of which are shown in FIG. 1, and motion sensor 108, consider a detailed illustration in FIG. 3. Generally, physiological sensors 106 are capable of detecting blood pressure, blood volume, skin color, displacement and so forth at one or more regions of a patient. Physiological sensors 106 may include a radar emitter and receiver, a standard RGB (red, green, blue) camera sensor, a monochrome sensor, a hyperspectral sensor, a stereoscopic sensor, a structured light sensor, a pressure sensor, an ultrasonic sensor, an electrical sensor (e.g., electrocardiograph (ECG) or an impedance cardiograph (ICG)), a reflective or transmissive PPG sensor, an audio sensor, or combinations of multiple sensors. Example emitters for sensing include one or a combination of nearly any of the electromagnetic spectrum in various forms, such as a combination of sources such as uniform, infrared, tangential, modulated/coded, or coherent (e.g., laser).

Motion sensors 108 are capable of detecting movement of patient 102. This may include movement from as small as a chest movement from a heartbeat or breathing and a slight shiver from being cold, to as large a physical movement as walking, moving arms, standing or sitting, lying down or getting up, and so forth. Some of physiological sensors 106 are also capable of sensing movement, such as sensors 106-1, 106-3, 106-4, 106-5, 106-6, and 106-7. Further, in some cases motion sensor 108 is specialized to determine movement, such as some types of gesture sensors used in gaming systems, SONAR systems, and so forth.

For example, motion sensor 108 can be an electromagnetic sensor capable of capturing motion data as a signal in an optical, radio-frequency, or infrared bandwidth. Motion sensor 108 may capture multiple images over a short time frame to enable chronological synchronization on a sub-millisecond range, or at least multiple images within a single time period smaller than a smallest time period of a cardiovascular event. By so doing, physiological signals for even a single heartbeat can be altered to improve a portion of the signal.

Consider, by way of example, pressure and electrical-sensing mat 106-2 and the motion-sensing capabilities of a camera, e.g., motion sensor 108 or sensors 106-3, 106-5, or 106-6. Patient 102 stands on the mat, which records physiological signals during a cardiovascular event (a heartbeat) of patient 102 through a pressure sensor sufficient to generate a BCG. Motion sensor 108 captures motion data as a video during the cardiovascular event. Both sensors include some sort of timing marker or are received at a same time. Motion module 212 of FIG. 2 determines, based on the video, physical movements of patient 102 leaning toward a mirror and thus putting more weight on the balls of her feet than on her heels, among other changes. Signal-quality module 214 receives the physiological signals from pressure and electrical-sensing mat 106-2, and then chronologically synchronizing the physical movement to the physiological signals. Signal-quality module 214 then alters the physiological signals or portions thereof based on the physical movement. Cardiovascular-function module 216 may then determine a hemodynamic characteristic for the heartbeat, such as a pressure wave, blood pressure, or pressure-volume loop. If the physical movement is too large or disruptive, signal-quality module 214 may down-weight or delete the physiological signal. If the physical movement is useful diagnostically, signal-quality module 214 may instead annotate the physiological signal for later analysis, trending with similar movements, and so forth.

Physiological sensors 106 and motion sensor 108 may also have a fixed position or consist of one or more mechanical targeting platforms or those that simply move due to being part of a mobile device. These sensors may also be separated into physically and spatially distinct devices capable of monitoring the body from multiple view angles or observing different regions of the body. Thus, one of these sensors may capture an image indicating blood volume at two different regions of patient 102.

In more detail, physiological sensor 106 can be one or a combination of various devices, whether independent, integral with, or separate but in communication with computing device 110. Eight examples are illustrated in FIG. 3, including hyperspectral sensor 106-1, pressure and electrical-sensing mat 106-2, color, displacement, and movement sensor 106-3 (e.g., a camera of computing device 110), structured-light or stereoscopic sensor system 106-4, optic sensor 106-5 of laptop 110-5, a wearable color, displacement, and movement sensor 106-6, which is part of computing spectacles 110-4, radar lamp 106-7, and ultrasonic bathtub 106-8.

Hyperspectral sensor 106-1 is capable of capturing images in an ultraviolet, visible, or infrared optical wavelength. Images recording these wavelengths can be used to determine various changes in blood movement or as calibration signals to detect changes in illumination or patient movement. In some cases blood perfusion and oxygen content can be ascertained, thereby further enabling robust measurement of cardiac function. Due to differential wavelength absorption between human tissue and blood, a hyperspectral sensor can also be used to penetrate the skin to map out veins and arteries to target closer examination for displacement and other measurements.

As noted in part above, pressure and electrical-sensing mat 106-2 is configured to measure the arrival times of cardiac electrical signals (e.g., ECG), cardiac generated forces (e.g., BCG), and cardiac driven blood flow pulsatility (e.g., PPG). The combination of these can sense a pulse-wave velocity of patient 102's blood. This pulse-wave velocity is a measure of a patient's cardiovascular health.

The signal-to-noise ratio of the signals from pressure and electrical-sensing mat 106-2 can be improved through synchronization with the other sensors, including motion sensor 108 of FIG. 1, to perform correlation techniques such as ensemble averaging and artifact rejection techniques through motion compensation. Further, the modules can synchronize physiological sensors 106 to enhance the processing of the physiological signals based on patient movement. Assume that motion sensor 108 or some other sensor 106 detects or determines physical movement of patient 102. With this movement known, signal-quality module 214 alters, compensate, and/or selectively weight the physiological signals gathered by pressure and electrical-sensing mat 106-2.

Structured-light sensor system 106-4 is capable of projecting structured light at patient 102 and sensing, often with two or more optical sensors, the projected structured light on patient 102 effective to enable capture of images having surface information. This surface information can be used to calculate depth and surface changes for a region of patient 102, such as skin, another organ, or other structure. These changes can be highly accurate, thereby indicating small vibrations and other changes in an organ or structure caused by the cardiovascular system, and thus how that system is operating. Structured-light sensor system 106-4 can, alternatively, be replaced with or supplemented with a targeted, coherent light source for more-accurate displacement measurements. This may include LIDAR (e.g., “light radar” or the process measuring distance by illuminating a target with a laser and analyzing light reflected from the target), laser interferometry, or a process of analyzing light speckle patterns produced by a coherent light on a skin's surface through optical tracking, which enables detection of very small skin displacements. These signals can be used a physiological signals or as motion data or both.

Radar lamp 106-7 is configured to reflect radiation from human tissue to measure heart rate, respiration rate, and skeletal movement, to name just three examples. Ultrasonic bathtub 106-8 is configured to generate high-frequency sound waves and to evaluate an echo from those waves. This echo is received at one or more sensors and the time interval between sending and receiving can be measured. These echoes enable analysis of internal body structures. In some cases, acoustic impedance of a two-dimensional cross-section of tissue can be measured, which can measure current heath or a health trend of the measured tissue. Blood flow, tissue movement, blood location, and three-dimensional measurements of structures can also be made. Non-active (no sound waves generated, just receiving sensors) can also be used, though accuracy and robust measurements are more difficult to achieve.

Some of these physiological sensors 106 capture images with sufficient resolution and at sufficient shutter speeds to show detailed colors and displacement, and thus enable determination of mechanical movements or vibrations. These mechanical movements and mechanical vibrations are sufficient to determine a ballistocardiogram (BCG) showing patient 102's cardiac function. Other sensing manners, such as color change or skin displacement in a different region of a patient's body, can be used to establish motion frequency bands to amplify, as well as a timing reference for aggregating multiple heartbeat measurements to improve accuracy of a BCG motion. This BCG information can also be used to provide reference timing information about when a blood pressure pulse leaves the left ventricle and enters the aorta, which combined with the other measurements across the body allow for more-precise estimates of pulse transit times and pulse-wave velocities.

While the BCG signal indicates the timing of the aortic valve, the timing of the atrial valve can be monitored by tracking atrial pressure waveforms visible in the external or internal jugular. This also allows the opportunity to detect atrial fibrillation by detecting missing atrial-pressure pulses. Additionally, aortic-wall stiffness has proven prognostic value in predicting cardiovascular morbidity and mortality. Measuring the pulse-transit time from the start of ejection from the left ventricle into the aorta and up the carotid allows an estimate of that aortic stiffness as well as trending of changes in that stiffness. Thus, determination of arterial-wall stiffness can made independent of blood pressure measurements.

In more detail, physiological sensors 106 are configured to capture sufficient information for the techniques to determine blood asymmetries and other cardiac function, including a pulse-wave velocity of patient 102's blood. This pulse-wave velocity is a measure of a patient's arterial health. In healthy arteries, the pulse-wave velocity is low due to the elasticity of the arteries but, as they harden and narrow, the pulse-wave velocity rises. As blood pressure increases and dilates the arteries, the walls become stiffer, increasing the pulse-wave velocity. While a particular pulse-wave velocity as a snapshot in time may or may not accurately indicate cardiovascular health (e.g., a one-time test at a doctor's office), a change in this pulse-wave velocity (that is, a trend), can be an accurate measure of a change in patient 102's cardiovascular health. If a positive trend, this can reinforce patient 102's healthy habits and, if negative, encourage changes to be made.

Cardiac-related measurements of a patient can include a patient's skin color sufficient to determine a photo-plethysmogram. This PPG measures variations in a size or color of an organ, limb, or other human part from changes in an amount of blood present in or passing through it. These colors and color variations in a patient's skin can show heart rate and efficiency.

Many of these physiological sensors 106 are non-invasive and even completely obscure to a patient. This often results in physiological signals that include errors, noise, and so forth. As noted, the techniques described herein alter those physiological signals to better permit accurate health conditions or trends for patients.

Returning to FIG. 3, physiological sensor 106 or motion sensor 108 may have various computing capabilities, though it may instead be a low-capability device having little or no computing capability. Here physiological sensor 106 or motion sensor 108 includes one or more computer processors 302, computer-readable storage media (CRM) 304, measurement element 306, and a wired or wireless transceiver 308 capable of receiving and transmitting information (e.g., to computing device 110).

Measurement element 306 may include various different sensors, from optics, radar, pressure, movement, acceleration, and so forth. Examples includes ultrasonic, pressure, and simple or complex cameras, such as those having low or high shutter speeds, low or high frame rates, low or high resolutions, and having or not having non-visible imaging capabilities.

Computer-readable storage media 304 includes sensor manager 314 and sync-management module 316. Sensor manager 314 is capable of processing physiological signals and recording and transmitting physiological signals, as well as receiving or assigning appropriate time markers by which to mark or compare the time of various captured images. These time markers can later be used by modules of computing device 110 to compare physical movement of a patient with a portion of physiological signal.

Sensor manager 314 and cardiovascular-function module 216 may also calibrate measurement element 306 through use of an external sensor. This can aid in calibrating skin colors or displacements to a calibration color or displacement, or even to a cardiac function, such as to a blood pressure or pulse-wave velocity. Thus, assume that one of physiological sensors 106 captures images for two regions while a blood pressure between those regions is also measured through a different device, thereby enabling more-accurate determination of cardiac functions for the physiological sensor and for that patient. Other potential calibration sensors include, but are not limited to, ECG, conventional BCG, digital stethoscopes, ultrasound, and the like. Another example is the use of an external blood pressure meter to calibrate the pulse wave velocity over time to determine long-term changes in arterial-wall stiffness by separating arterial stiffness due to blood pressure versus that due to the dilation by blood pressure.

These and other capabilities, as well as ways in which entities of FIGS. 1-3 act and interact, are set forth in greater detail below. These entities may be further divided, combined, and so on. The environment 100 of FIG. 1 and the detailed illustrations of FIGS. 2 and 3 illustrate some of many possible environments capable of employing the described techniques.

### Example Methods

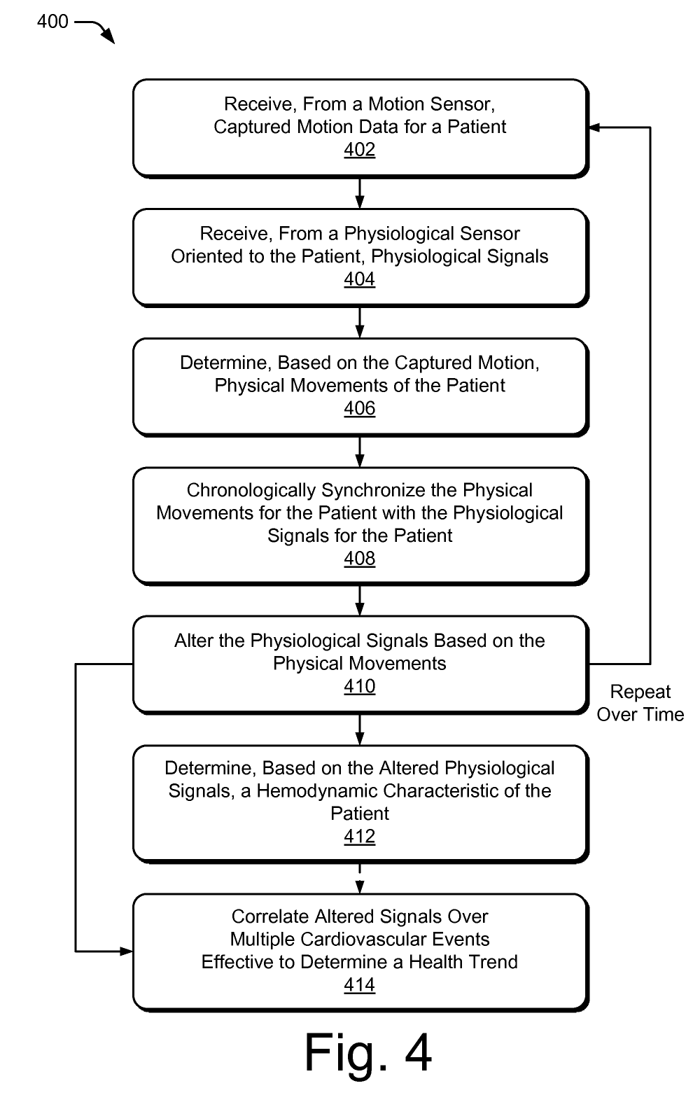

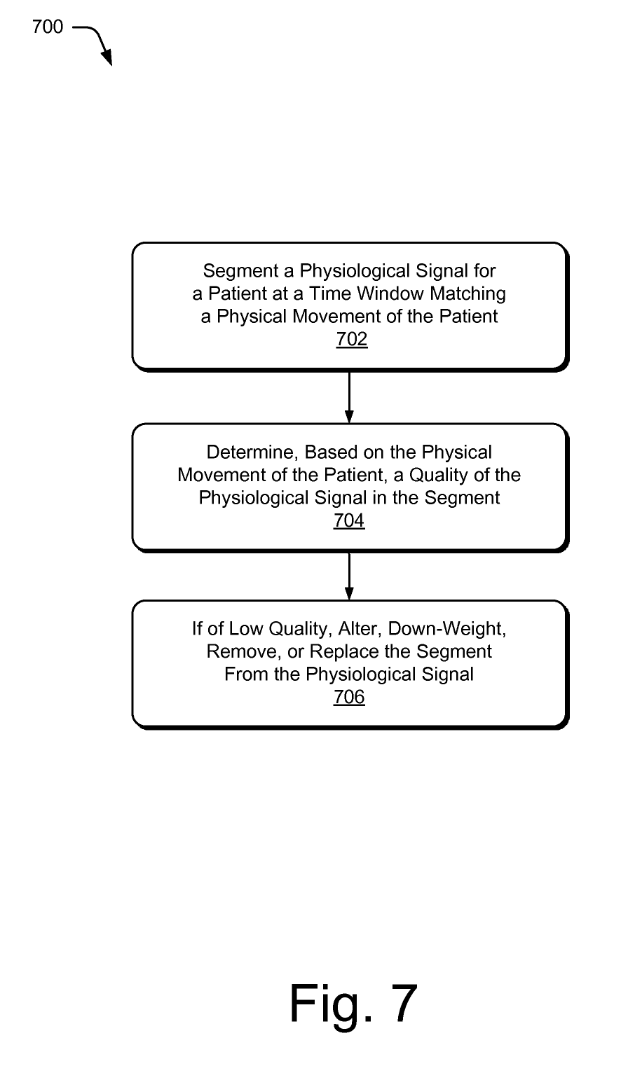

FIGS. 4 and 7 depict methods 400 and 700, which alter physiological signals based on patient movement. These methods are shown as sets of blocks that specify operations performed but are not necessarily limited to the order or combinations shown for performing the operations by the respective blocks. In portions of the following discussion, reference may be made to environment 100 of FIG. 1 and entities detailed in FIGS. 2 and 3, reference to which is made for example only. The techniques are not limited to performance by one entity or multiple entities operating on one device.

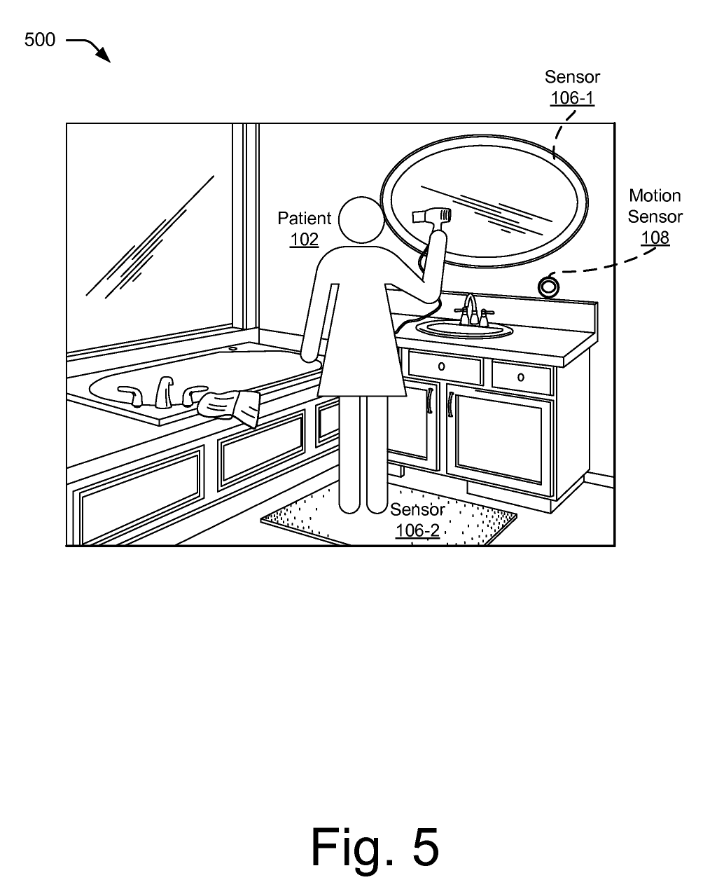

At 402, captured motion data for a patient is received from a motion sensor. By way of an ongoing example, consider FIG. 5, which shows a sensing milieu 500 in which patient 102 stands on pressure and electrical-sensing mat 106-2 and in front of hyperspectral sensor 106-1 and motion sensor 108. Here motion sensor 108 captures motion data for patient 102 and passes it to a computing device, such as server 110-2 of FIG. 2.

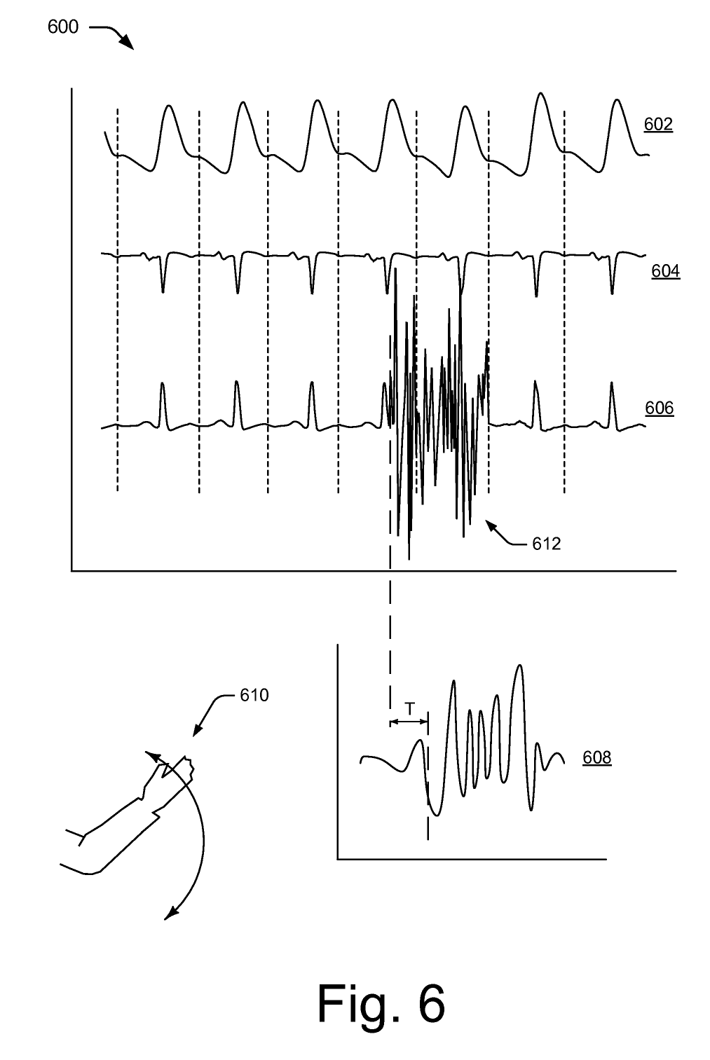

At 404, physiological signals are received from a physiological sensor oriented to the patient. Continuing the example, assume hyperspectral sensor 106-1 captures a physiological signal for patient 102, shown in FIG. 6 in physiological signal chart 600, at PPG waveform 602. Pressure and electrical-sensing mat 106-2 captures two physiological signals for patient 102, ECG signal 604 and BCG signal 606.

At 406, physical movements of the patient are determined based on the captured motion data. As noted, motion sensor 108 captures motion data, here shown in a simplified form at motion data 608 in FIG. 6. Based on this motion data 608, motion module 212 determines a physical motion of patient 102, here shown in picture form (though a signal form can instead be used), at arm motion 610.

At 408, the physical movements for the patient are chronologically synchronized with the physiological signals for the patient. Here signal-quality module 214 synchronizes arm motion 610 by synchronizing motion data 608 to physiological signals 602, 604, and 606 by a time period T illustrated in FIG. 6.

At 410, the physiological signals are altered based on the physical movements. As noted, signal-quality module 214 is configured to alter physiological signals 114, such as a respiration physical movement used to improve a blood pressure physiological signal or a heart rate physical movement being used to alter a physiological signal for a skin color or skin volume change. In the ongoing example, signal-quality module 214 alters a portion 612 that likely includes noise or a signal error of BCG signal 606. In this example the alteration to BCG signal 606 is simply to remove it and corresponding cardiac cycles of physiological signals 602 and 604 from use to determine a hemodynamic characteristic. These other physiological signals 602 and 604 may instead by used during those cardiac cycles, however. In this case many cardiac cycles and physiological signals are available, reducing any need to rely on the cardiac cycles having the noise or error. As noted, however, signal-quality module 214 may instead replace, annotate, or down-weight portion 612.

At 412, a hemodynamic characteristic of the patient is determined based on the altered physiological signals. Concluding the example of FIGS. 5 and 6, portion 612 is removed, along with corresponding portions of physiological signals 602 and 604. Cardiovascular-function module 216 then determines the hemodynamic characteristic for patient 102.

At 414, the altered signals from multiple cardiovascular events are correlated effective to determine a health trend. As noted with the Repeat Over Time arrow, multiple captured motion data and physiological signals can be attained and altered physiological signals determined. With these multiple, altered physiological signals, a trend can be determined by correlating these altered signals. By so doing, a health trend over extended periods, such as days, weeks, or even years can be determined. Or, for shorter periods where more than one signal for one type of event is desired, multiple altered physiological signals enable determination of better graphs representing heart health, such as a pressure-volume loop.

Signal-quality module 214 and cardiovascular-function module 216 may also use physiological signals where the alteration is an annotation indicating a particular physical movement. This particular physical movement can be correlated to the altered physiological signals of multiple same or similar physical movements to provide a robust hemodynamic measurement or trend for the particular physical movement. Examples of useful movements are provide above. In the example of FIGS. 5 and 6, arm motion 610, and similar other movements, may be useful in assessing some hemodynamic characteristics, such as determining blood pressure when an arm is in a high position over patient 102's heart, especially when using an imaging sensor, as vessels of patient 102's arm show blood pressure based on the arm's position relative to patient 102's heart.

FIG. 7 illustrates method 700, in which physiological signals are altered based on patient movement, including through determining that a segment of the physiological signal is corrupt.

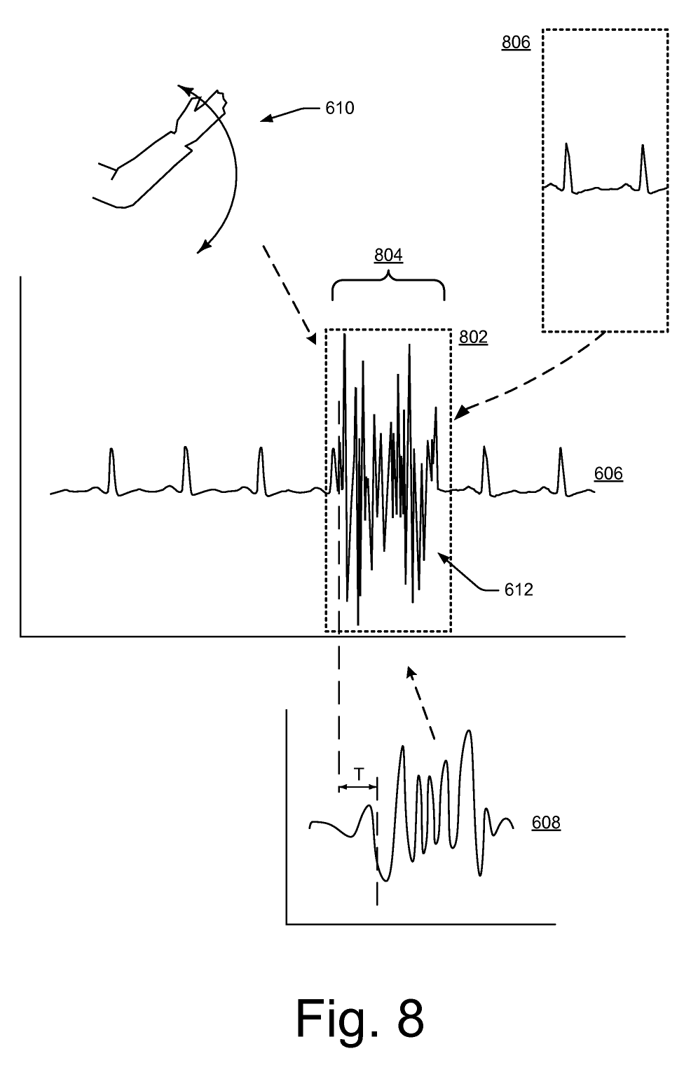

At 702, a physiological signal for a patient is segmented at a time window, the time window matching a physical movement of the patient. Consider, for example, FIG. 8, which includes portions of FIG. 6. Here time window 802 is determined to match arm motion 610 and motion data 608, thereby segmenting BCG signal 606 to include segment 804.

At 704, a quality of the physiological signal during the segment is determined based on the physical movement of the patient during the segment. This can be performed before, after, or concurrent with segmenting the physiological signal above. Thus, on determining that a patient is running, rapidly moving her arm and holding a loud hair dryer, driving, and so forth, a physiological signal measured during that physical movement can be determined to be of low quality. The information about the physical movement may indicate that this type of movement renders the physiological signal less accurate, less useful, or otherwise corrupt.

This determination of low quality can be dispositive, causing the physiological signal of the segment to be altered or discarded. In some cases, however, further analysis is performed. For example, assume that signal-quality module 214 determines, based on the segment of the physiological signal and information about the physical movement, that the physiological signal in the segment is of low quality. After, concurrent, even prior to this determination, assume that signal-quality module 214 determines that the physiological signal of the segment resides outside of possible parameters for the physiological signal or its corresponding hemodynamic function, either generally, or for the patient based on prior data about the patient.

At 706, responsive to the segment of the physiological signal being determined to be of low-quality, the segment is altered, down-weighted, removed, or replaced in the physiological signal. In some cases, altering the segment includes matching the segment with a previously calculated template, finding a prefix that minimizing a cost of the matching, aligning boundaries of the segment, and updating the physiological signal with the altered segment.

Consider a segment that indicates, on its face, a heart rate of 490 beats per minute in a healthy adult. This would be considered by signal-quality module 214 to be corrupt or at least suspect, which can then be confirmed based on the physical movement. To alter this, an immediately prior heart rate of 82 beats per minute, or after of 84 beats per minute, can replace the segment. Further, other physiological signals may be used that, for the same physical movement, do not appear to be corrupt. These signals in turn indicate that a heartbeat of 85 is appropriate. To replace the segment, a previously calculated template for the patient for a heartbeat of 85 can be used, this is shown with alteration 806 of FIG. 8, which is then used to replace segment 804.

Signal-quality module 214 may instead down-weight the segment effective to reduce, in a combination of physiological signals of multiple similar cardiovascular events, a weight of the segment. By so doing, the weight of the segment is reduced in determining a hemodynamic characteristic for the patient during the physical movement, such as to rely on physiological signals 602 and 604 instead of 606 as shown in FIG. 6.

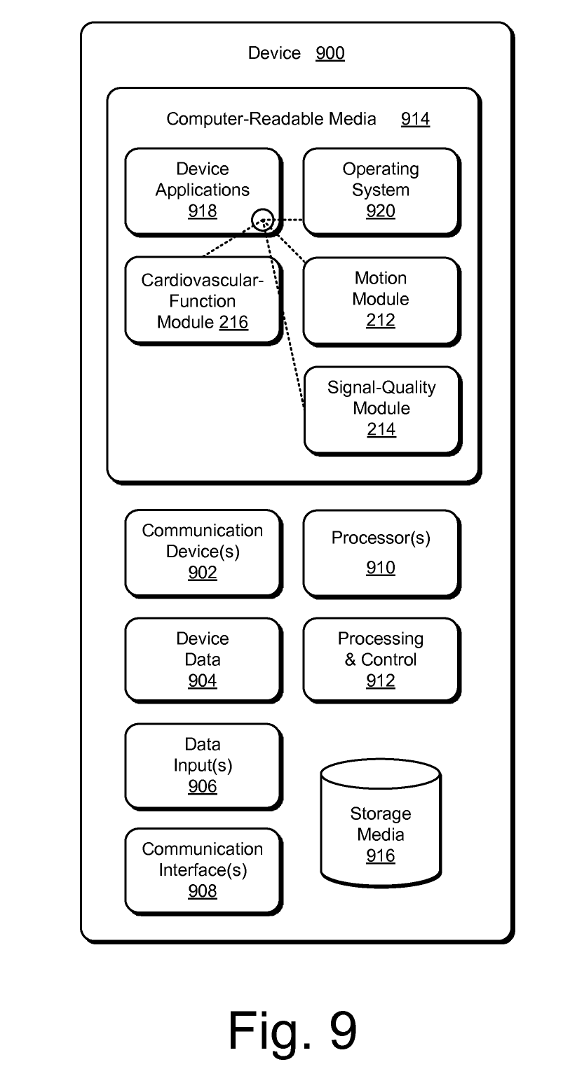

The preceding discussion describes methods relating to assessing cardiac function and altering physiological signals based on patient movement for a human cardiovascular system. Aspects of these methods may be implemented in hardware (e.g., fixed logic circuitry), firmware, software, manual processing, or any combination thereof These techniques may be embodied on one or more of the entities shown in FIGS. 1-3, 5, 6, and 9 (computing system 900 is described in FIG. 9 below), which may be further divided, combined, and so on. Thus, these figures illustrate some of the many possible systems or apparatuses capable of employing the described techniques. The entities of these figures generally represent software, firmware, hardware, whole devices or networks, or a combination thereof.

### Example Computing System

FIG. 9 illustrates various components of example computing system 900 that can be implemented as any type of client, server, and/or computing device as described with reference to the previous FIGS. 1-8. In embodiments, computing system 900 can be implemented as one or a combination of a wired and/or wireless wearable device, System-on-Chip (SoC), and/or as another type of device or portion thereof. Computing system 900 may also be associated with a user (e.g., a patient) and/or an entity that operates the device such that a device describes logical devices that include users, software, firmware, and/or a combination of devices.

Computing system 900 includes communication devices 902 that enable wired and/or wireless communication of device data 904 (e.g., received data, data that is being received, data scheduled for broadcast, data packets of the data, etc.). Device data 904 or other device content can include configuration settings of the device, media content stored on the device, and/or information associated with a user of the device. Media content stored on computing system 900 can include any type of audio, video, and/or image data, including complex or detailed results of cardiac function determination. Computing system 900 includes one or more data inputs 906 via which any type of data, media content, and/or inputs can be received, such as human utterances, user-selectable inputs (explicit or implicit), messages, music, television media content, recorded video content, and any other type of audio, video, and/or image data received from any content and/or data source.

Computing system 900 also includes communication interfaces 908, which can be implemented as any one or more of a serial and/or parallel interface, a wireless interface, any type of network interface, a modem, and as any other type of communication interface. Communication interfaces 908 provide a connection and/or communication links between computing system 900 and a communication network by which other electronic, computing, and communication devices communicate data with computing system 900.

Computing system 900 includes one or more processors 910 (e.g., any of microprocessors, controllers, and the like), which process various computer-executable instructions to control the operation of computing system 900 and to enable techniques for, or in which can be embodied, such as altering physiological signals based on patient movement. Alternatively or in addition, computing system 900 can be implemented with any one or combination of hardware, firmware, or fixed logic circuitry that is implemented in connection with processing and control circuits, which are generally identified at 912. Although not shown, computing system 900 can include a system bus or data transfer system that couples the various components within the device. A system bus can include any one or combination of different bus structures, such as a memory bus or memory controller, a peripheral bus, a universal serial bus, and/or a processor or local bus that utilizes any of a variety of bus architectures.

Computing system 900 also includes computer-readable media 914, such as one or more memory devices that enable persistent and/or non-transitory data storage (i.e., in contrast to mere signal transmission), examples of which include random access memory (RAM), non-volatile memory (e.g., any one or more of a read-only memory (ROM), flash memory, EPROM, EEPROM, etc.), and a disk storage device. A disk storage device may be implemented as any type of magnetic or optical storage device, such as a hard disk drive, a recordable and/or rewriteable compact disc (CD), any type of a digital versatile disc (DVD), and the like. Computing system 900 can also include a mass storage media device 916.

Computer-readable media 914 provides data storage mechanisms to store device data 904, as well as various device applications 918 and any other types of information and/or data related to operational aspects of computing system 900. For example, an operating system 920 can be maintained as a computer application with computer-readable media 914 and executed on processors 910. Device applications 918 may include a device manager, such as any form of a control application, software application, signal-processing and control module, code that is native to a particular device, a hardware abstraction layer for a particular device, and so on.

Device applications 918 also include any system components, modules, or managers to implement the techniques. In this example, device applications 918 include motion module 212, signal-quality module 214, and cardiovascular-function module 216.

### Conclusion

Although embodiments of techniques for, and apparatuses enabling, altering physiological signals based on patient movement have been described in language specific to features and/or methods, it is to be understood that the subject of the appended claims is not necessarily limited to the specific features or methods described. Rather, the specific features and methods are disclosed as example implementations of these techniques.

1. A system comprising:
one or more computer processors;
a physiological sensor configured to measure a physiological characteristic of a patient;
a motion sensor configured to capture motion data for the patient, the motion data comprising physical movement of the patient separate from the physiological characteristic of the patient;
one or more computer-readable media having instructions stored thereon that, responsive to execution by the one or more computer processors, perform operations comprising:
determining, based on the captured motion data, the physical movement of the patient;
receiving a physiological signal from the physiological sensor;
synchronizing chronologically the determined physical movement of the patient with the received physiological signal; and
altering the received physiological signal based on the determined physical movement.

2. The system of claim 1, wherein the altering the received physiological signal down-weights the received physiological signal based on determining the physical movement comprises a physical movement indicative of a non-steady state or a non-resting state of the patient.

3. The system of claim 1, wherein the altering the received physiological signal removes the received physiological signal or a portion thereof based on determining the physical movement comprises a disruptive physical movement of the patient.

4. The system of claim 1, wherein the altering the received physiological signal alters the received physiological signal for a particular cardiovascular event based on determining the physical movement comprises a respiration of the patient, the respiration having a known effect on cardiovascular events.

5. The system of claim 1, wherein the altered received physiological signal is effective to measure a hemodynamic characteristic of the patient.

6. The system of claim 5, wherein the hemodynamic characteristic includes a pulse-wave velocity or pressure waves representing blood flow through an artery or vein of the patient.

7. The system of claim 1, the operations further comprising determining, prior to the altering, a portion of the received physiological signal that is likely to include noise or signal error, and wherein the altering of the received physiological signal is based on the chronological synchronization matching a portion of the physical movement for the patient determined to cause noise or signal error with the determined portion of the received physiological signal that is likely include noise or signal error.

8. The system of claim 1, the operations further comprising annotating the received physiological signal based on determining the physical movement comprises one of a set of previously determined physical movements useful in assessing cardiovascular health.

9. The system of claim 1, wherein the motion sensor is an electromagnetic sensor and the captured motion data is captured as a signal in an optical, radio-frequency, or infrared bandwidth.

10. The system of claim 1, wherein the motion sensor is camera configured to capture multiple images over a single time period smaller than a time period of a cardiovascular event.

11. The system of claim 1, wherein the received physiological signal includes an electrocardiograph (ECG), a ballistocardiogram (BCG), a blood pressure, or a photo-plethysmogram (PPG).

12. The system of claim 1, wherein the physiological sensor comprises a pressure sensor and the physiological characteristic comprises pressure and wherein the pressure sensor senses the patient during a cardiovascular event, the motion sensor comprises a camera, the captured motion data comprises a video captured during the cardiovascular event, and the altering the received physiological signal is effective to down-weight noise in the received physiological signal.

13. A computer-implemented method comprising:
receiving, from a physiological sensor configured to measure a physiological characteristic of a patient, a physiological signal;
receiving, from a motion sensor configured to capture motion data for the patient, captured motion data comprising physical movement of the patient separate from the physiological characteristic of the patient;
chronologically synchronizing the physical movement for the patient with the received physiological signal for the patient; and
altering the received physiological signal based on the physical movement.

14. The computer-implemented method as described in claim 13, further comprising determining, based on the altered received physiological signal, a hemodynamic characteristic of the patient.

15. The computer-implemented method as described in claim 13, wherein the captured motion data and the received physiological signal is for a first cardiovascular event and further comprising repeating the method for a second cardiovascular event to provide a second altered received physiological signal and correlating the first and second altered received physiological signals for the first and second cardiovascular events, respectively, effective to determine a health trend for the patient.

16. The computer-implemented method as described in claim 13, wherein the physical movement is a respiration of the patient and altering the received physiological signal based on the physical movement alters the received physiological signal to improve a blood pressure measurement.

17. The computer-implemented method as described in claim 13, wherein altering the received physiological signals annotates the received physiological signals, the annotation indicating a particular physical movement, and further comprising correlating the altered received physiological signal with a second altered received physiological signal having a same particular physical movement effective to provide a hemodynamic measurement of the patient for the particular physical movement.

18. A computer-implemented method comprising:
segmenting a physiological signal for a patient at a time window, the time window matching a physical movement of the patient, the physiological signal sensed by a physiological sensor and the physical movement sensed by a motion sensor, the physical movement separate from a physiological characteristic sensed by the physiological sensor;
determining, based on the physical movement of the patient, a quality of the physiological signal in the segment; and
responsive to the quality being determined to be a quality indicative of signal corruption, altering, down-weighting, removing, or replacing the segment of the physiological signal.

19. The computer-implemented method as described in claim 18, wherein altering, down-weighting, removing, or replacing the segment replaces the segment with a previously calculated template.

20. The computer-implemented method as described in claim 18, wherein altering, down-weighting, removing, or replacing the segment down-weights the segment effective to reduce, in a combination of physiological signals of multiple similar cardiovascular events, a weight of the segment in determining a hemodynamic characteristic or a trend in the hemodynamic characteristic.
